(19) United States
(12) Patent Application Publication
(10) Pub. No.: US 2021/0322099 A1
(43) Pub. Date: Oct. 21, 2021

(54) VEIN ABLATION

(71) Applicant: THE WHITELEY CLINIC LIMITED, Guildford (GB)

(72) Inventors: Mark Steven Whiteley, Guildford (GB); Charles Esteves Peretra, Guildford (GB)

(21) Appl. No.: 17/235,028

(22) Filed: Apr. 20, 2021

(30) Foreign Application Priority Data
Apr. 21, 2020 (GB) 2005780.8

(51) Int. Cl. A61B 18/24

(52) U.S. Cl. CPC A61B 18/24; A61B 2018/2222

(57) ABSTRACT

The present invention relates to a method of ablating a vein in a subject. The method comprises introducing an ablation member into the vein, wherein the ablation member has an external diameter of less than 5 French Gauge (FG); and emitting thermal energy with a power output of less than 5.5 Watts from the ablation member to thereby ablate the vein.

The present invention relates to vein ablation, and particularly, although not exclusively, to apparatuses and methods used for carrying out vein ablation on a subject. In particular, the invention extends to apparatus and methods used for the ablation of prominent forehead veins of a subject.

### BACKGROUND

A number of individuals suffer from prominent forehead veins. As illustrated in FIG. 1, these veins are typically located in the centre of the forehead, running from the scalp down to the top of the nose between the eyebrows. The number of these veins can vary, with some individuals having one prominent vein whilst others may have two or more. The anatomy of these veins can also differ between individuals, with veins being vertical, diagonal, or appearing to branch. Although these veins can start from various positions on the scalp, they all end up at the top of the nose between the eyebrows.

Despite these veins being present in everybody, they are only prominent and noticeable in some individuals. In those who are affected, the nature of these veins causes a very noticeable cosmetic impact, with a vertical line or bulge attracting attention and creating embarrassment for the individual suffering from it. In particular, these individuals say it ruins photographs and makes them very uncomfortable socially. Due to the fact that these veins tend to dilate when smiling, laughing, bending forwards, lying down, when hot, after exercise, or with alcohol, they are often dilated in social situations or during holidays.

Previous treatments have involved physical removal of the veins. However, these methods are associated with a high failure rate and result in scarring. Sclerotherapy has also been used, whereby a solution, or a combination of a solution and a gas to make a foam, is injected into the enlarged vein, causing it to shrink and close. However, this treatment also has a high failure rate and is not recommended due to the risk of embolization of the sclerosant through the veins and possibly into the brain. Other treatment attempts have involved external laser surgery. However, this is often unsuccessful and damages the skin because these veins are too large and too deep for successful external laser treatment.

There is, therefore, the need for an improved apparatus and method of treatment for removing these prominent forehead veins from patients.

### SUMMARY OF THE INVENTION

The inventors have designed a new apparatus comprising a slim radial laser and a short introduction cannula positioned using a Seldinger approach, for use in treating patients with prominent forehead veins.

According to a first aspect of the invention, there is provided a vein ablation apparatus comprising:
a thermal energy source, which is configured to provide thermal energy at a power output of less than 5.5 Watts; and
an ablation member having an external diameter of less than 5 French Gauge (FG), wherein the ablation member is connected or connectable to the thermal is energy source and is configured to allow insertion into a vein and to transfer thermal energy from the thermal energy source to a wall of the vein.

Advantageously, the apparatus comprises an ablation member with a small diameter, which the inventors have found is sized to allow it to be introduced into a vein in a subject's forehead. The inventors have found that a power of less than 5.5 Watts is sufficient to ablate a forehead vein. Additionally, the low power prevents damage to the skin and surrounding structures.

Preferably, the ablation member has an external diameter of less than 4.5 FG, less than 4 FG or less than 3.5 FG. Preferably, the ablation member has an external diameter of between 1 and 5 FG, more preferably between 1.5 and 4.75 FG or between 2 and 4.5 FG, and most preferably between 2.5 and 4 FG or between 3 and 3.5 FG.

The ablation member may comprise an optical fibre. The optical fibre may be a radial fibre, a bare fibre, a forward firing fibre, a jacket-tipped fibre, a two ring radial fibre, a three ring radial fibre or a combination thereof. In an embodiment where the optical fibre is a jacket-tipped fibre, a first end of the optical fibre may be encased in a cylinder. The cylinder may comprise or consist of a ceramic or metal. Advantageously, this prevents the optical fibre from touching the vein wall. Preferably, the optical fibre is a radial fibre. Advantageously, this radial fibre emits thermal energy in a 360 degree arc around the end of the fibre.

A first end of the optical fibre may define a cone. Advantageously, in embodiments where a laser is used, this ensures that the laser beam emerges from the tip radially and therefore, laser light is emitted in a 360 degree range into the surrounding vein wall.

Preferably, the first end of the optical fibre is configured to emit light at an angle between 60 and 120° or between 70 and 110° relative to the axis of the fibre more preferably between 85 and 95° or between 87 and 93° relative to the axis of the fibre, or between 89 and 91° to the axis of the fibre. Most preferably, the first end of the optical fibre is configured to emit light at an angle of about 90° relative to the axis of the fibre.

In another embodiment, the first end of the optical fibre defines a divergent cone so that laser light is emitted in a forward direction. In this embodiment, the first end of the optical fibre is configured to emit light at an angle between 1 and 89° relative to the axis of the fibre, more preferably between 5 and 60° relative to the axis of the fibre and most preferably between 10 and 40° to the axis of the fibre.

The ablation member may comprise a cover member configured to cover at least the first end of the optical fibre. The cover member is preferably transparent. Advantageously, this allows transmission of the laser beam. The cover member may comprise a plastic.

Preferably, the cover member comprises a first end. The first end of the cover member may define a pointed or rounded shape. Advantageously, the first end of the cover member better enables the ablation member to be inserted into a vein.

The first end of the optical fibre may be disposed towards or substantially adjacent to the first end of the cover member. The ablation member may comprise an air pocket defined within the cover member. The air pocket may be disposed towards or substantially adjacent to the first end of the cover member. The first end of the optical fibre may be disposed in the air pocket.

Preferably, at least a portion of the cover member is removable from the ablation member. Preferably, the at least a portion of the cover member comprises the first end of the cover member. Preferably, the at least a portion of the cover member comprises any portion of the cover member which may contact a subject during use of the apparatus. Advantageously, the at least a portion of the cover member may be disposed and replaced to ensure that the apparatus is sterile.

The apparatus may comprise a cannula, with a first end and a second end defining a channel there between. The cannula may have an external diameter of less than 20 FG, more preferably less than 15 FG or less than 10 FG, and most preferably less than 8 FG or less than 6 FG and an internal diameter which is at least as large as the external diameter of the ablation member. Accordingly, the optical member may be reversibly inserted through the cannula. Preferably, the cannula has an external diameter of between 3 and 6 FG, more preferably between 3.5 and 5.5 FG, and most preferably between 4 and 5 FG.

The apparatus may comprise a needle, with a first and second end defining a channel there between. The needle may comprise a hypodermic needle. The needle may have a diameter gauge of between 5 and 40 G. In a first embodiment, the needle may have a gauge of between 10 and 30 G, more preferably between 12 and 25 G and most preferably between 15 and 21 G. Advantageously, the needle can accommodate a guide wire. In an alternative embodiment, the needle may have a gauge between 15 and 30 G or between 17 and 27 G, more preferably between 19 and 25 G or between 21 and 23 G. Advantageously, the needle can accommodate a bare optical fibre.

The thermal energy source may be configured to deliver thermal energy at a power output of less than 5 Watts, more preferably less than 4.5 Watt or less than 4 Watts. The thermal energy source may be configured to deliver thermal energy at a power output of between 0.5 and 5.5 Watts or between 1 and 5 Watts, more preferably between 1.5 and 4.5 Watts or between 2 and 4 Watts. Most preferably, the thermal energy source is configured to deliver thermal energy at a power output of 3 Watts. Preferably, the power output is measured at a first end of the ablation member, and more preferably at a first end of the optical fibre.

The thermal energy source may be configured to deliver thermal energy of between 18 and 24 joules per centimetre of vein, more preferably between 19 and 23 joules per centimetre of vein, and most preferably between 20 and 22 joules per centimetre of vein.

The thermal energy source may be a laser source, a radiofrequency radiation source, an electromagnetic radiation source or a steam source. In some embodiments, the thermal energy source is a laser source.

The laser source may be a diode laser, a crystal laser or a liquid crystal laser. Preferably, the laser source is a diode laser.

The cannula may have a length of less than 4.5 cm or less than 4 cm, preferably, less than 3.5 cm, less than 3 cm, less than 2.5 cm, less than 2 cm or less than 1.5 cm. Preferably, the length of the introduction cannula is between 0.1 and 5 cm, more preferably between 0.25 and 4.5 cm, between 0.5 and 4 cm or between 0.75 and 3.5 cm, and most preferably between 1 and 3 cm.

Preferably, the vein ablation apparatus further comprises a dilator comprising a tip, wherein an external diameter of the tip increases across a length thereof, wherein a first end of the tip defines the smallest external diameter and a second end of the tip defines the largest external diameter. The length of the tip may be less than 2 cm, more preferably less than 1.5 cm or less than 1.25 cm, and most preferably less than 1.1 cm. The length of the tip may be between 0.25 and 2 cm, more preferably between 0.5 and 1.5 cm or between 0.75 and 1.25 cm, and most preferably between 0.9 and 1.1 cm.

The dilator may comprise an elongate portion extending between the second end of the tip and a second end of the dilator. The elongate portion may have an external diameter sized to allow it to be reversibly disposed in the channel of the cannula. Preferably, the elongate portion has an external diameter of less than 5 FG, less than 4.5 FG, less than 4 FG or less than 3.5 FG. Preferably, the elongate portion has an external diameter of between 1 and 5 FG, more preferably between 1.5 and 4.75 FG or between 2 and 4.5 FG, and most preferably between 2.5 and 4 FG or between 3 and 3.5 FG. The external diameter of the second end of the tip may be substantially the same as an external diameter of the elongate portion. Preferably, the length of the elongate member is substantially the same as the length of the cannula.

The second end of the dilator may comprise a stop configured to prevent the second end of the dilator from passing through the channel of the cannula.

Preferably, the dilator defines a channel therein extending between the first and second ends thereof. The channel may be sized to receive a guide wire therein.

The apparatus may further comprise a guidewire.

According to a second aspect of the present invention, there is provided a method of ablating a vein in a subject, the method comprising:
introducing an ablation member into the vein, wherein the ablation member has an external diameter of less than 5 French Gauge (FG); and
emitting thermal energy with a power output of less than 5.5 Watts from the ablation member to thereby ablate the vein.

Preferably, the ablation member is as defined in relation to the first aspect.

The vein is preferably disposed in the subject's forehead.

Initially, the method may comprise placing the subject in a head down position, where the subject's heart and legs are above the subject's forehead. This position may be the Trendelenburg position.

The method may comprise applying pressure to the cheeks of the patient prior to introducing the ablation member. The pressure may be applied by the patient pushing on their own cheeks. Advantageously, this increases the size of the vein.

Prior to introducing the ablation member, the method may comprise making an incision in the skin of the subject.

The method may comprise introducing a needle comprising a channel into the vein. The incision may be made towards or adjacent to the top of the subject's forehead and/or above the subject's hairline.

The incision is made at an angle between 5° and 90° relative to the subject's skin, more preferably at an angle between 10° and 70° or between 20° and 60° relative to the subject's skin and most preferably at an angle between 30° and 50° relative to the subject's skin. In a preferred embodiment, the incision is made at an angle between 35° and 45° relative to the subject's skin.

The inventors have found that an angled incision is advantageous as this increases the overlap of skin and therefore the surface area to heal back together. Accordingly, scarring is reduced as the skin almost acts like a “trapdoor” rather than a gap. It also means that closure is easier. In cases where it is necessary to actively bring the skin together, then the upper edge naturally falls onto the lower edge and can be stuck in place with a plaster or glue.

The needle may be inserted through the incision at an angle between 10° and 90° relative to the subject's skin, more preferably at an angle between 20° and 70° or between 30° and 60° relative to the subject's skin and most preferably at an angle between 40° and 50° relative to the subject's skin. In a preferred embodiment, the needle is inserted at an angle of about 45° relative to the subject's skin.

Alternatively, the method may not include making an incision in the patient's skin. Instead, introducing a needle comprising a channel into the vein, thereby creating an aperture in the patient's skin. Accordingly, the needle may simply be inserted through the patient's skin. The needle may be inserted at an angle between 10° and 90° relative to the subject's skin, more preferably at an angle between 20° and 70° or between 30° and 60° relative to the subject's skin and most preferably at an angle between 40° and 50° relative to the subject's skin. In a preferred embodiment, the needle is inserted at an angle of about 45° relative to the subject's skin.

It may be appreciated that an angle relative to the subject's skin may mean relative to a plane defined by the top surface of the subject's skin at the point of the incision.

The ablation member may be introduced into the vein by passing it through the channel of the needle. In this embodiment, the needle may be a hypodermic needle. The hypodermic needle may have a gauge between 15 and 30 G or between 17 and 27 G, more preferably between 19 and 25 G or between 21 and 23 G.

Alternatively, a guidewire may be passed through the channel of the needle, and thereby be introduced into the vein, and the needle may then be removed from the vein. It may be appreciated that this method could be referred to as a Seldinger approach. The needle may have a gauge of between 10 and 30 G, more preferably between 12 and 25 G and most preferably between 15 and 21 G.

The method may comprise disposing a cannula over a dilator comprising a channel. The method may comprise feeding the guidewire into the channel of the dilator, and thereby dilating the incision or aperture and introducing the dilator and cannula into is the vein. The method may then comprise removing the dilator and guidewire from the vein. The dilator and cannula may be as defined in the first aspect.

Introducing an ablation member into the vein may comprise passing the ablation member through the cannula into the vein. The method may comprise advancing the ablation member along the vein.

Preferably, the ablation member is advanced along the vein until a first end of the ablation member is disposed substantially adjacent to the top of the nose and/or between the eyebrows of the subject.

The method may comprise introducing the needle into the vein using direct vision or an imaging technique such as ultrasound guidance.

The method may comprise administering at least one of a local anaesthetic, bicarbonate and/or adrenaline to the subject. Preferably, the at least one of local anaesthetic, bicarbonate and/or adrenaline is administered to the forehead of the subject. Preferably, the at least one of local anaesthetic, bicarbonate and/or adrenaline is administered adjacent to the vein. Preferably, the at least one of local anaesthetic, bicarbonate and/or adrenaline is administered after the cannula and/or the ablation member are introduced into the vein. Preferably, local anaesthetic, bicarbonate and/or adrenaline is not administered before the cannula and/or the ablation member are introduced into the vein. The inventors have found that local anaesthetic can cause the vein to spasm making cannulation more difficult. It may be appreciated that the amount of the at least one of local anaesthetic, bicarbonate and/or adrenaline to be administered may vary depending upon the length of the vein to be treated. Preferably, the volume of the at least one of local anaesthetic, bicarbonate and/or adrenaline administered is between 0.1 and 50 ml per cm of vein to be treated, more preferably between 0.25 and 25 ml, between 0.5 and 10 ml, between 0.75 and 7.5 ml or between 1 and 5 ml per cm of vein to be treated, and most preferably between 1.25 and 3 ml or between 1.5 and 2.5 ml per cm of vein to be treated. Most preferably, the volume of the at least one of local anaesthetic, bicarbonate and/or adrenaline administered is about 2 ml per cm of vein. For instance, between 10 and 20 ml of local anaesthetic solution may be administered to a 7 cm vein. It may be appreciated that adrenaline may be alternatively referred to as epinephrine.

The method may comprise locating the subject in a head-up position, where the subject's heart and legs are below the subject's forehead. This position may be the reverse-Trendelenburg position. The method may comprise locating the subject in a head-up position after the cannula and/or the ablation member are introduced into the vein. The method may comprise locating the subject in a head-up position after the local anaesthetic, bicarbonate and/or adrenaline is administered.

The method may comprise cooling the skin over the vein. The method may comprise cooling the skin by disposing a cold pack thereon. A cold pack may comprise a cooling substance. For instance, the cold pack may comprise ice. The cooling substance may be disposed within a container, such as a bag. Alternatively, the method may comprise cooling the skin using a cold air blower. Cooling the skin using a cold air blower is particularly advantageous because it does not push the skin onto the laser, which would increase the risk of burns and skin marking. Instead, it allows the skin to be cooled whilst keeping it maximally away from the laser and the vein which is being burnt. The method may comprise cooling the skin over the vein after the cannula and/or the ablation member are introduced into the vein. The method may comprise cooling the skin over the vein after the local anaesthetic and/or bicarbonate is administered. The method may comprise cooling the skin over the vein after the subject has been located in a head-up position.

The thermal energy may be provided as a laser beam, radiofrequency radiation, electromagnetic radiation or steam. In a preferred embodiment, the thermal energy is provided as a laser beam.

The wavelength of the laser beam may be between 750 and 2500 nm, and more preferably between 1000 and 2000 nm. In one embodiment, the wavelength of the laser beam may be between 1100 and 1800 nm, between 1200 and 1700 nm or between 1300 and 1600 nm, and most preferably is between 1400 and 1550 nm, between 1450 and 1500 nm or between 1460 and 1480 nm. In one embodiment, the wavelength of the laser beam is about 1470 nm. In an alternative embodiment, the wavelength of the laser beam may be between 1600 and 2300 nm, between 1700 and 2200 nm, between 1800 and 2100 nm or between 1850 and 2000 nm, and most preferably is between 1900 and 1980 nm, between 1920 and 1960 nm or between 1930 and 1950 nm. In a is preferred embodiment, the wavelength of the laser beam is about 1940 nm. Advantageously, a laser with a wavelength of 1940 nm has a high affinity for water, which reduces the spread of heat.

Preferably, the thermal energy is emitted from the ablation member at a power output of less than 5.5 Watts, less than 4.5 Watts or less than 4 Watts. Preferably, the thermal energy is emitted from the ablation member at a power output of between 0.5 and 5.5 Watts or between 1 and 5 Watts, more preferably between 1.5 and 4.5 Watts or between 2 and 4 Watts. Most preferably, the thermal energy is emitted from the ablation member at a power output of 3 Watts. Preferably, the power output is measured at a first end of the ablation member.

The method may comprise moving the ablation member along the vein as the thermal energy is emitted. Preferably, the ablation member, and optionally also the cannula, is moved out of the vein as the thermal energy is emitted. The ablation member may be moved along the vein at a speed of between 1 and 20 s/cm, more preferably between 2 and 15 s/cm or between 3 and 12 s/cm, more preferably between 4 and 11 s/cm or between 5 and 10 s/cm and most preferably between 6 and 8 s/cm.

It may be appreciated that the power output and the speed that the ablation member is moved along the vein will affect the amount of energy which is delivered. Accordingly, the power output and the speed may be optimised to deliver a desired amount of energy to a portion of vein. Preferably, the method comprises delivering between 10 and 30 joules of energy per centimetre of vein, between 15 and 25 joules per centimetre of vein or between 18 and 24 joules per centimetre of vein, more preferably between 19 and 23 joules per centimetre of vein, and most preferably between 20 and 22 joules per centimetre of vein.

In another embodiment, the method may comprise holding the ablation member stationary in the vein as the thermal energy is emitted. The thermal energy may be emitted for a predetermined interval. Preferably, the ablation member is then moved and re-positioned in the vein before subsequently emitting the thermal energy. The method may comprise repeating one or more of the steps recited above to treat a further branch of the vein.

The method may comprise sealing the incision. The incision may be sealed with glue or a plaster.

All features described herein (including any accompanying claims, abstract and drawings), and/or all of the steps of any method or process so disclosed, may be combined with any of the above aspects in any combination, except combinations where at least some of such features and/or steps are mutually exclusive.

### BRIEF DESCRIPTION OF THE SEVERAL VIEWS OF THE DRAWINGS

For a better understanding of the invention, and to show how embodiments of the same may be carried into effect, reference will now be made, by way of example, to the accompanying Figure, in which:

FIG. 1A shows a front view of a subject's head showing the typical anatomy of their forehead veins;

FIG. 1B shows a front view of a further subject's head showing the typical anatomy of their forehead veins;

FIG. 1C shows a front view of a still further subject's head showing the typical anatomy of their forehead veins;

FIG. 2 is a schematic side view of an embodiment of a vein ablation apparatus according to the invention, comprising an introduction cannula, a laser fibre and a dilator;

FIG. 3 is a cross-sectional side view of a section of a subject's forehead comprising a vein;

FIG. 4 shows a subject placing their hands on their cheeks so that they are resting next to the nose, creating pressure so that the forehead veins become more prominent—a manoeuvre previously published as the “Whiteley-Smith” sign;

FIG. 5 illustrates a subject on an operating table in the Trendelenburg position;

FIG. 6A illustrates a first step in a method of ablating forehead veins using the vein ablation apparatus shown in FIG. 2. As shown in the Figure, in the first step a hollow needle is inserted through the skin of the patient and passed into the vein to be treated;

FIG. 6B illustrates a second step in the method of ablating forehead veins, where a guidewire is passed through the channel of the needle;

FIG. 6C illustrates a third step in the method of ablating forehead veins, where the needle is removed;

FIG. 6D illustrates a fourth step in the method of ablating forehead veins, where a dilator is disposed with a cannula, and both are passed over the guidewire;

FIG. 6E illustrates a fifth step in the method of ablating forehead, where the dilator slightly enlarges the tract originally produced by the needle and enables the cannula to be placed within the vein;

FIG. 6F illustrates a sixth step in the method of ablating forehead veins, where the dilator and guidewire are removed leaving the cannula in position;

FIG. 6G illustrates a seventh step in the method of ablating forehead veins, where a slim laser fibre is passed through the cannula, down the vein, and is positioned with the tip at the top of the nose between the eyebrows;

FIG. 6H illustrates an eighth step in the method of ablating forehead veins, where local anaesthetic is injected around the vein; and

FIG. 7 shows cross-sectional views of a skin incision made using a hollow needle at both a 90 degree (A) and 45 degree angle (B) to the surface of the skin.

### DETAILED DESCRIPTION OF THE PREFERRED EMBODIMENTS

### Examples

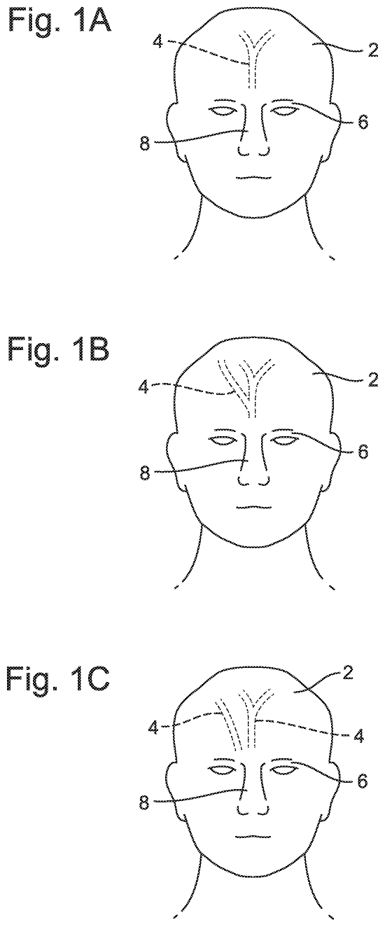

Referring first to FIG. 1, there are shown front views of three different subjects' heads 1a, 1b and 1c, showing the typical anatomy of their forehead veins 4.

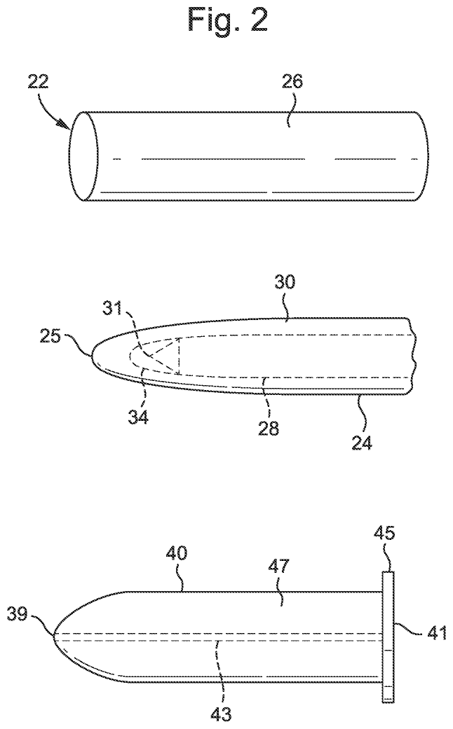

An apparatus 22 for ablation of prominent forehead veins 4 is shown in FIG. 2, comprising an introduction cannula 26 and a laser fibre 24.

As shown in FIG. 2, the laser fibre 24 is an elongate member extending between a first end 25 and a second end (not shown). Due to the size of forehead veins, the laser fibre 24 would have a relatively small diameter, of about 3-4 FG (French Gauge).

The laser fibre 24 comprises a plastic coating 30 extending between the first end 25 and the second end. The plastic coating 30 coating defines a tip defining rounded or pointed shape at the first end, configured to aid insertion of the laser fibre 24. The plastic coating further defines an air bubble 34, disposed within or adjacent to the tip of the plastic coating 30.

The laser fibre 24 further comprises an optical fibre 28 and a source of thermal energy, such as a laser source (not shown). The optical fibre 28 is an elongate member disposed within the plastic coating and extends between a second end (not shown) and a first end 31. The second end of the optical fibre 28 is substantially adjacent to the second end of the laser fibre 24 and is connected or connectable to the laser source. The first end 31 of the optical fibre 28 is disposed in the air bubble 34. In some examples, the first end 31 of the optical fibre is in the shape of a cone 31, ensuring that the laser beam emerges from the tip radially. Accordingly, laser light may be emitted in a 360 degree range around the circumference of the fibre into the surrounding vein wall. This is enhanced by the shape of the first end 25 of the laser fibre 24 and the air bubble 34.

In one embodiment, the laser used for this technique is a diode laser. However, in other embodiments, the laser could be a crystal laser or liquid crystal laser. In addition, fibres other than a radial fibre could be used to perform this technique. For example, this method may be performed using a bare fibre, a forward firing fibre, a jacket-tipped fibre, a two ring radial fibre or a three ring radial fibre.

In other embodiments, the source of thermal energy source may not be a laser source. For example, a radiofrequency source may be used to generate radiofrequency radiation for the ablation of forehead veins. Alternatively, electromagnetic radiation or steam sclerotherapy may be used.

The cannula 26 is an elongate, tubular member, and is configured to enter a forehead vein of a subject. That is, the cannula may be dimensioned such that it may be inserted into a subject's vein. The standard cannula used for veins of the leg needs to pass through the skin and various layers of subcutaneous fat at an angle. Consequently, the cannula for ablation of leg veins is approximately 10 cm long, but can range from 5 cm to 50 cm or more. However, as a forehead vein can be quite short, in the region of 2-7 cm, and is very near the surface of the skin, the standard length cannula used for leg vein ablation may not be appropriate for this treatment. Therefore, the cannula 26 need only be very short, and can be in the region of 1-3 cm. The cannula 26 and laser fibre 24 are sized such that the laser fibre 24 may be reversibly disposed within the cannula 26. Accordingly, the internal diameter of cannula may be 3-4 FG. The external diameter of the cannula may be 3-6 FG. In contrast, the standard cannula used in leg vein ablation has an internal diameter of approximately 6 FG.

The apparatus 22 may further comprise a dilator 40. The dilator 40 comprises an elongate member 47 extending between a first end 39 and a second end 41. A channel 43 extends through the elongate member 47 between the first and second ends 39, 41. The elongate member 47 is sized such that it may be reversibly disposed within the cannula 26. Accordingly, it may have a diameter of 3-4 FG. The second end 41 of the elongate member 40 comprises a handle 45 sized to prevent the second end 41 of the elongate member 47 from passing through the cannula 26. The first end 39 of the elongate member 39 defines a pointed end configured to be inserted into and enlarge an aperture in a body. The first end 39 of the dilator 40 need only stand proud from a first end of the cannula 26 by approximately 1 cm. Conversely, a dilator used for leg veins should stand proud of a cannula by about 2-3 cm. If the dilator 40 is too long it could be difficult to position the cannula 26 within the forehead vein 4. Accordingly, the length of the elongate member 40 may be about 1 cm longer than the length of the cannula, i.e. it could be about 2 to 4 cm long.

When working with humans or animals, it is essential to maintain a sterile environment. Particularly, if an instrument is not sterile, the bacteria that may have collected on the instrument can be transferred to a tissue, cause infection. Therefore, medical instruments need to be easy to sterilise, or designed for one-time use. Sterilising instruments typically involves heating the instrument using steam in an autoclave. Clearly, this is not appropriate for instruments having electrical components. Methods of sterilization also include using gamma radiation and Ethylene Oxide (EtO) treatment, but these are costly and labour-intensive.

Therefore, in one embodiment, the cannula may be disposable. At least a portion of the plastic coating 30 comprising the tip may also be disposable. This prevents wastage of the optical fibre 28, which has not come into contact with a human or animal. The cannula and/or portion of the plastic coating may be encased in a sterile air-tight package prior to use.

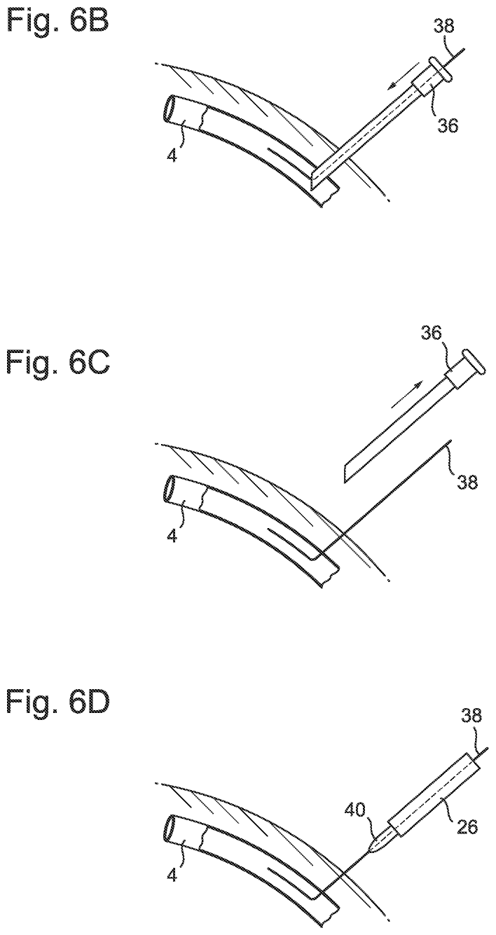

To perform the procedure, a subject 18 is placed on the operating table 20. Although it is possible to do this with the patient supine, it is advantageous to place the patient in a head down position, known as the Trendelenburg position (FIG. 5). In this position, the heart and legs are above the forehead veins, making the veins more obvious and tense as they fill with venous blood. Consequently, the veins do not collapse when the initial part of the procedure is performed.

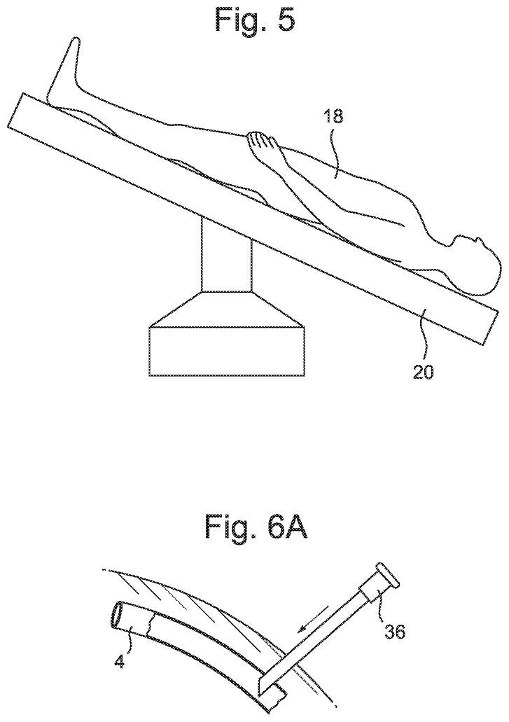

Alternatively or additionally, if, while the patient is sitting or standing, the central forehead veins are not visible, the patient may place their hands 16 on their cheeks so that they are resting next to the nose 8, as seen in FIG. 4. This pressure blocks draining of the facial veins and as a result, the forehead veins 4 become more prominent.

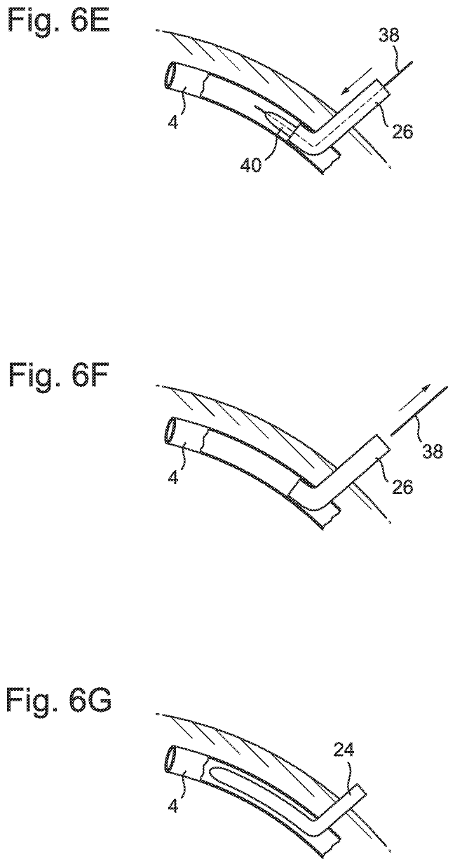

FIG. 6 illustrates a method of ablating forehead veins 4 using the vein ablation apparatus 22 and the Seldinger technique. It is possible to gain access to the veins with a variety of techniques. A direct intravenous cannulation can be performed using a cannula and needle combination (i.e. a standard intravenous cannulation). However, a Seldinger technique, as described below, is likely to be more successful.

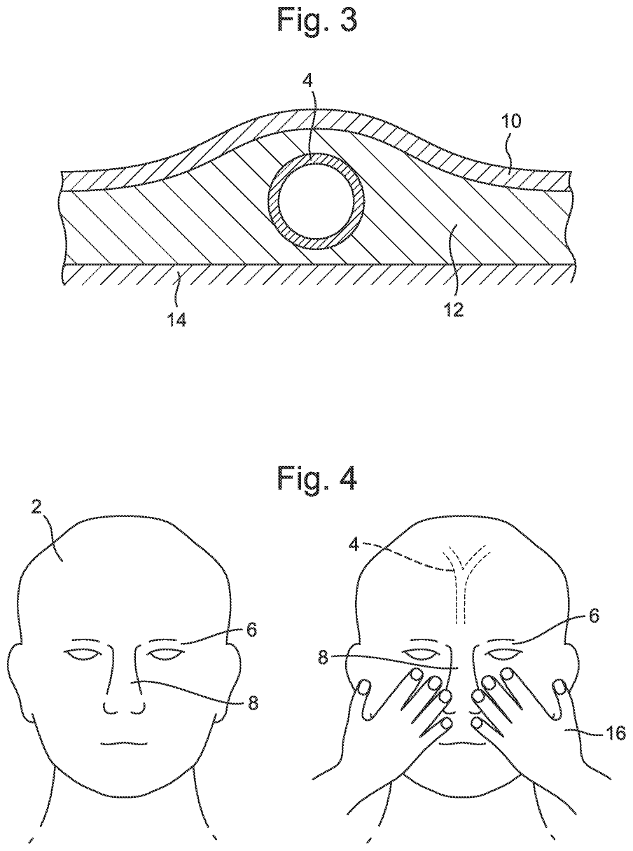

A subject's skin 10 is prepared and any sterile drapes are put in position. FIG. 3 illustrates a cross-sectional side view of a forehead vein, in which the vein 4 is positioned within the frontalis muscle 12, that is located between the skin 10 and the skull bone 14. As shown in FIG. 6a, a hollow needle 36 is inserted through the skin and passed into the vein to be treated 4. As the vein of the forehead is short and small, it is optimal to use a very thin needle between 21-31 gauge. The needle 36 is inserted as high up on the scalp and as close to or above the hairline as is possible. The needle 36 may be inserted using direct vision or an imaging technique such as ultrasound guidance.

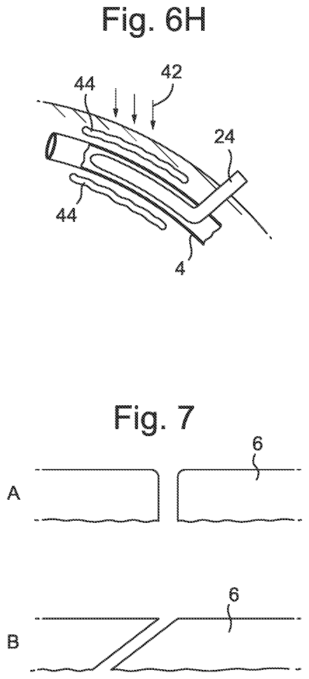

It may be advantageous to make a small skin incision at an angle to facilitate the passage of both the initial needle and the subsequent dilator. This angle is approximately 45° to the perpendicular and is slanted in the direction of the needle passing into the vein (FIG. 7). The incision is positioned this way because the skin of the forehead is tough. Making the slanted incision allows the needle and subsequent dilator to be passed directly into the vein without excessive resistance.

In addition, by making the incision at an angle to the perpendicular, healing is much improved and scars are far more likely to be cosmetically acceptable or even invisible. This is because an incision through the skin at an angle to the perpendicular, creates a very small surface area for healing, reducing the amount of scar tissue visible on the surface.

Alternatively, the inventors have found that it is possible to insert a dilator and cannula using the Seldinger technique through a single needle hole. Accordingly, a preliminary incision is not essential.

If a bare fibre or very thin laser fibre are being used, the Seldinger approach may be replaced by simple insertion of a hypodermic needle. A hypodermic needle, such as a 21 gauge needle, can be passed directly into the vein and a bare fibre or other similar very fine laser fibre can be passed directly through this hypodermic needle and into the vein.

Alternatively, if the Seldinger approach is followed, a guidewire 38 is then passed through the channel of the needle 36 (FIG. 6b), and once in place, the needle 36 is removed (FIG. 6c). Next, the dilator 40 is disposed with the cannula 26, and both are passed over the guidewire. The dilator 40 slightly enlarges the tract originally produced by the needle 36 (FIG. 6d) and enables the cannula to be placed within the vein 4 (FIG. 6e). Finally, the dilator 40 and the guidewire 38 are removed from the patient leaving the cannula 26 in position (FIG. 6f).

Once the cannula 26 is within the vein 4 to be treated, the slim laser fibre 24 is passed through the cannula 26, down the vein 4, and is positioned with the tip at the top of the nose between the eyebrows (FIG. 6g). There may be one vein or multiple veins that need treatment and so this process can be repeated for any number of veins that are being treated.

Once the cannula 26, and preferably also the laser fibre 24, are in place local anaesthetic 44 is injected around the veins (FIG. 6h). Bicarbonate can be added to the local anaesthetic to reduce pain and adrenaline (epinephrine), and to increase constriction of the vein around the fibre, thereby removing any venous blood still within the vein. A sufficient volume is used to separate the veins from the skin and skull, using the concept of “tumescence”. This protects the skin, skull and any other surrounding structures from thermal damage.

At this stage, it is advantageous to tip the patient into a “head-up” (reverse-Trendelenburg) position, further reducing the chance of any blood being in the vein during treatment. In addition, it is also useful to cool the skin 42 overlying the vein during treatment to reduce the risk of any skin damage or burns.

Once the patient is in an optimum position with tumescence in place and cooling of the skin, if required, the laser is fired producing continuous output energy at 1470 nm or 1940 nm and a constant power output of 2-4 Watts, preferably 3 Watts.

A power of between 2-4 Watts, and more specifically 3 Watts is optimal, with lower powers not causing enough damage to the vein and higher powers causing damage to the skin or surrounding structures. Comparatively, a power of 6-12 Watts is required in the ablation of leg veins.

The use of a laser with a wavelength of 1940 nm is advantageous because the chromophore of this wavelength is water. It is possible to use other wavelengths, however it is clear that those which use water as the chromophore have a significant advantage over those that use haemoglobin or other chromophores. Additionally, since a wavelength of 1940 nm has a higher affinity for water, heat does not spread as far out from the vein, resulting in a safer treatment as the risk of skin burns is reduced.

The optimal power output of 3 Watts is selected with the aim of delivering power between 18-24 joules per centimetre of vein, and more preferably between 20-22 joules per centimetre of vein. Energy transference to the vein wall is determined by the pullback speed. A pullback speed of seven seconds per centimetre is optimal. However, this may vary depending on the size and circumference of the vein and therefore, pullback can range from 3-12 seconds per centimetre.

Once the whole length of the vein or each vein has been treated, the skin is closed with glue or a simple plaster.

Advantages of the apparatus of the invention reside in the ability to carry out endovenous ablation of much smaller veins of the forehead, compared with the apparatus that is currently used for endovenous ablation of much larger veins of the leg. The shorter introduction cannula (1-3 cm) of the apparatus allows entry into small forehead veins, positioned close to the surface of the skin. In contrast, the apparatus for leg vein ablation comprises a much longer introduction cannula which would be very difficult to position within a vein of the forehead. In addition, the small diameter laser fibre of the apparatus can enter much smaller veins of the forehead and emit laser energy at a much lower power. This is very advantageous, since the leg vein ablation apparatus has a laser fibre with a diameter that is too large for entering forehead veins and emits laser energy at a much higher power which would cause damage to the skin of the forehead.

What is claimed is:

1. A method of ablating a vein in a subject, the method comprising:
introducing an ablation member into the vein, wherein the ablation member has an external diameter of less than 5 French Gauge (FG); and
emitting thermal energy with a power output of less than 5.5 Watts from the ablation member to thereby ablate the vein.

2. The method according to claim 1, wherein the vein is disposed in the subject's forehead.

3. The method according to claim 2, wherein the ablation member is advanced along the vein until a first end of the ablation member is disposed substantially adjacent to the top of the nose and/or between the eyebrows of the subject.

4. The method according to claim 1, wherein the method initially comprises placing the subject in a head down position, where the subject's heart and legs are above the subject's forehead.

5. The method according to claim 1, wherein the method comprises:
introducing a needle comprising a channel into the vein, thereby creating an aperture in the patient's skin;
passing a guidewire through the channel of the needle, and thereby introducing it into the vein, and then removing the needle from the vein;
disposing a cannula over a dilator comprising a channel;
feeding the guidewire into the channel of the dilator, and thereby dilating the aperture and introducing the dilator and cannula into the vein;
removing the dilator and guidewire from the vein; and
introducing the ablation member into the vein comprises passing the ablation member through the cannula into the vein.

6. The method according to claim 1, comprising administering at least one of a local anaesthetic, bicarbonate and/or adrenaline to the subject adjacent to the vein.

7. The method according to claim 1, comprising locating the subject in a head-up position, where the subject's heart and legs are below the subject's forehead, after the ablation member is introduced into the vein.

8. The method according to claim 1, comprising moving the ablation member along the vein as the thermal energy is emitted at a speed of between 1 and 20 s/cm, or between 2 and 15 s/cm, or between 3 and 12 s/cm, or between 4 and 11 s/cm, or between 5 and 10 s/cm, or between 6 and 8 s/cm, and/or holding the ablation member stationary in the vein as the thermal energy is emitted.

9. The method according to claim 1, wherein the thermal energy is provided as a laser beam.

10. The method of claim 9, wherein the laser beam has a wavelength between 750 and 2500 m, optionally between 1600 and 2300 nm.

11. The method according to claim 1, wherein the thermal energy is emitted from the ablation member at a power output of between 0.5 and 5.5 Watts, between 1 and 5 Watts, between 1.5 and 4.5 Watts or between 2 and 4 Watts, and/or wherein the power output is measured at a first end of the ablation member.

12. The method of claim 1, wherein the method comprises delivering between 10 and 30 joules of energy per centimetre of vein.

13. The method according to claim 4, wherein the needle is introduced into the vein using an imaging technique, optionally wherein the imaging technique is ultrasound guidance.

14. The method according to claim 4, wherein the needle is inserted at an angle between 10° and 90°, between 20° and 70°, between 30° and 60°, or between 40° and 50° relative to the subject's skin.

15. The method according to claim 4, wherein the needle has a gauge of between 10 and 30 G, or between 12 and 25 G, or between 15 and 21 G.

16. The method according to claim 6, wherein the local anaesthetic, bicarbonate and/or adrenaline is administered after the cannula and/or the ablation member are introduced into the vein.

17. The method according to claim 6, wherein the volume of the at least one of local anaesthetic, bicarbonate and/or adrenaline administered is between 0.1 and 50 ml, between 0.5 and 10 ml, between 1 and 5 ml, between 1.25 and 3 ml, or between 1.5 and 2.5 ml per cm of vein to be treated.

18. The method according to claim 1, wherein the method comprises cooling the skin over the vein after the ablation member is introduced into the vein, optionally wherein the method comprises cooling the skin using a cold air blower.

19. The method according to claim 1, wherein the ablation member has an external diameter of between 1 and 5 FG, between 1.5 and 4.75 FG, between 2 and 4.5 FG, between 2.5 and 4 FG or between 3 and 3.5 FG.

20. The method according to claim 4, wherein the cannula comprises a first end and a second end defining a channel there between, wherein the cannula has an external diameter of less than 20 FG, an internal diameter which is at least as large as the external diameter of the ablation member and a length of less than 4.5 cm.
